(19) United States
(12) Patent Application Publication
(10) Pub. No.: US 2004/0215401 A1
(43) Pub. Date: Oct. 28, 2004

(54) Computerized analysis of forensic DNA evidence

(76) Inventors: Dan Edward Krane, Xenia, OH (US); Travis Edward Doom, Fairborn, OH (US); Michael Lee Raymer, Yellow Springs, OH (US); Oscar Nicolas Garcia, Beavercreek, OH (US)

Correspondence Address: Killworth, Gottman, Hagan & Schaeff, L.L.P., Suite 500, One Dayton Centre, Dayton, OH, 45402-2023, US

(21) Appl. No.: 10/423,188

(22) Filed: Apr. 25, 2003

(51) Int. Cl. C12Q 1/68; G06F 19/00; G01N 33/48; G01N 33/50

(52) U.S. Cl. 702/20; 435/6

(57) ABSTRACT

A process and expert system for presenting an expert analysis of collected forensic DNA evidence in a form more suitable for human analysis is provided. The expert system accepts as input electronic forensic DNA data output of a conventional genetic analysis program, and automates the interpretation of such data according to accepted forensic DNA evidence standards. The resulting output of the expert system is the forensic DNA data and analysis summaries which are presented in a format useable to aid interpretation by both expert and non-expert humans, such as, for example, police officers, attorneys, judges, and juries.

### BACKGROUND OF THE INVENTION

The present invention relates generally to computerized analysis of experimental information, and particularly, but not exclusively, to computerized systems and methods for the expert analysis of forensic DNA evidence.

DNA analysis is an elaborate process. Once a DNA sample has been obtained, it is amplified using Polymerase Chain Reaction (PCR). PCR works as a sort of molecular duplicating machine, making copies of the DNA sample in a lab. An example of a commercially available amplifying system is the PowerPlex® Systems, Promega Corp. STRs are variable regions of DNA, which differ greatly between people making them an excellent form of identification. As the Short Tandem Repeat (STR) regions within a sample's human genomic DNA are amplified, they are marked with a dye, and placed in a genetic analyzer that separates them on the basis of their size. Conventional genetic analyzers use capillary electrophoresis to separate the STR segments by fragment size or alleles. A laser coupled with a photo-detector is then used to detect amplified fragments of different sizes. Examples of such conventional systems are the Applied Biosystems, Inc. (ABI) PRISM 310 Genetic Analyzer and the ABI PRISM 3100 DNA Sequencer.

Typically, the raw DNA sample or detection data from the genetic analyzer is recorded or stored on a computer readable medium, such as a compact disk (CD), as evidence. However, a CD of unprocessed detection data is not very useful because it requires further analysis. Accordingly, such detection data is then imported into an analysis program. The current standard in analysis software is Applied Biosystems' (ABI) GeneScan and GenoTyper analysis programs. The GeneScan program identifies the intensities corresponding to each of several different dyes in a sample that are present in the detection data and represents this data as a series of allele peaks in an electropherogram. This information regarding the allele peak's intensities for each dye in a lane is then analyzed using the GenoTyper program to identify and label each allele peak. Among other things, the GenoTyper program filters out peaks/bands caused by a technical artifact of the amplification process known as “stutter”, which may have been erroneously identified by the GeneScan program. Stutter peaks appear as progressively smaller peaks before each real or “true” allele peak. The GenoTyper program also arranges and presents allele peaks into groups and categories depending on their color and size.

In crimes where biological evidence is involved, DNA typing can be used to determine whether the evidence contains DNA that is consistent with that of known individuals. However, one particular problem is that DNA typing is difficult to understand and typically not in a format useable for non-expert human analysis, such as police, juries, judges, and legal counsel. Further acerbating the above problem is that conventional analyzers and analysis programs generate enormous amounts of data. In particular, the GenoTyper program produces output in the form of an electropherogram, a graph that looks similar to an EKG (see FIG. 1). The graph is mostly flat with peaks representing the presence of individual STR alleles. Currently, DNA experts analyze such electropherograms by eye to identify problems and to match or resolve multiple samples. A typical case can easily result in hundreds of separate output electropherograms, making analysis a sizable task. Accordingly, a significant amount of the expert's time is spent selecting the samples to analyze, setting up the correct parameters, performing the analysis, and saving and organizing the results. Furthermore, the standards and practices used by analysts differ widely. Accordingly, the massive volumes of data generated by these systems and programs, and the effective management and use of such information have created a number of very challenging problems.

Additional problems include errors in the automated DNA analysis process, problems with the DNA samples themselves, and analyst bias. Evidence technicians and laboratory personnel sometimes incorrectly handle, process, and label samples. Analysis bias and flexible standards have also caused labs to miss or disregard problems with the DNA output data in order to simplify the presentation and interpretation of results. DNA testing has great potential, but the analysis needs to be performed objectively in order to solve these analysis problems.

Accordingly, there is a need for a computer-implemented method and system which will organize and present the enormous amount of analysis data produced by conventional genetic analysis programs in a convenient fashion. What is also needed is a computer-implemented method and system which will analyze such DNA output data objectively to identify possible errors for further analysis, thereby minimizing an expert's time in sorting through volumes of such analysis data for possible problems.

### SUMMARY OF THE INVENTION

The above-mentioned needs are met by the present invention, which provides a process of presenting an expert analysis of collected forensic DNA evidence in a form more suitable for human analysis. The expert system of the present invention accepts as input the electronic forensic DNA output or analysis data of a conventional genetic analysis program, and automates the interpretation of such analysis data according to accepted forensic DNA evidence standards. The resulting output of the expert system is the forensic DNA data and analysis summaries which are presented in a format useable to aid interpretation by both expert and non-expert humans, such as, for example, police officers, attorneys, judges, and juries. An example of such summaries, include: an allelic summary table, which has a potential stutter in italics, victim alleles in red, defendant in blue, and others in black; frequency calculations; and procedural interpretations. The procedural interpretations may include providing preliminary data interpretations such as providing population statistics, peak height determinations, and highlighting possible analysis problems such as indications of: mixture; sample degradation; injection failure; marginal stutter indications; signal saturation; failed control reactions; peak height determination and the like. In addition to aiding in the understanding and interpreting complex forensic DNA evidence, other advantages of the present invention include providing faster, unbiased, and correct results, permitting analysis and handling of more forensic DNA evidence in less time.

According to a first aspect of the invention, a method for performing triage analysis of forensic DNA evidence on a computer is provided. The method comprises obtaining a signal produced by a sequence of electrophoretically separated DNA fragments from the forensic DNA evidence, the signal providing allele size and quantity information, and utilizing an expert system having a knowledge base and an inference engine to perform triage analysis on the DNA fragments using the signal. The inference engine executes at least one rule from the knowledge base for classifying the allele information. The expert system outputs summaries in a format useable to aid interpretation of the triage analysis.

According to a second aspect of the invention, software for configuring a computer system comprising a processor, and a memory device, for performing triage analysis of forensic DNA evidence is provided. The software comprises program instructions for the execution of a pre-processing routine which obtains as input a signal produced by a sequence of electrophoretically separated DNA fragments from the forensic DNA evidence, and prepares the signal for analysis. The software also permits the execution of an automated analysis routine comprising a knowledge base and an inference engine to perform triage analysis on the DNA fragments using the signal. The inference engine executes at least one rule from the knowledge base for classifying allele information provided in the signal. The software further permits the execution of a post-processing routine which creates summaries in a format useable to aid interpretation of the triage analysis.

According to a third aspect of the invention, an expert computer system for performing triage analysis of forensic DNA evidence is provided. The computer system comprises means to load electrophoretic data containing a plurality of alleles, means to format the data if necessary, means to analyze the data, and means to output results of analyzed data, the results providing indications of possible and/or probable problems corresponding to as well as correlations between particle alleles in the data.

These and other features and advantages of the invention will be more fully understood from the following description of some embodiments of the invention taken together with the accompanying drawings. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1a is an illustration of an electropherogram of graphed analysis data provided by a conventional genetic analysis program displaying the presence of alleles, shown as peaks, identified in inputted detection data from a conventional genetic analyzer. The graph is broken into three sections, representing three different chromosomal locations (loci) within the DNA of the human genome, wherein smaller to larger alleles are organized left to right, and the height of peak is proportional to the amount of DNA.

FIG. 1b is a “zoomed” electrograph having a lower relative fluorescent unit (RFU) cutoff value than that of the electropherogram of FIG. 1a, but based on the same inputted detection data from a conventional genetic analyzer, and showing smaller peaks more clearly, which may be evidence of an additional, minor contributor to the sample.

FIG. 2 is a diagrammatic illustration of an expert system in accordance with an embodiment of the present invention.

FIG. 3 is a flow chart of the presently disclosed method for analyzing DNA fragments separated electrophoretically.

FIG. 4 is an illustration of a graphic user interface according to an embodiment of the present invention.

FIG. 5 is an HTML navigation page providing an organized and convenient manner by which to review analysis data outputted by conventional genetic analysis programs.

FIG. 6 is a rule map for the DNA STR analysis that can be performed by the expert system according to an embodiment of the present invention.

FIG. 7 is sample output of a summary table outputted by the expert system according to an embodiment of the present invention.

FIG. 8 shows the order of the data processing steps conducted by the expert system.

FIG. 9 is an electropherogram illustrating a technical artifact known as a “spike” detected by the expert system of an embodiment of the present invention in the inputted analysis data received from conventional genetic analysis programs.

FIG. 10 is an electropherogram illustrating mixtures and degradation detected by the expert system according to an embodiment of the present invention.

### DETAILED DESCRIPTION OF THE PREFERRED EMBODIMENTS

The following detailed description of the preferred embodiments presents a description of certain specific embodiments to assist in understanding the claims. However, the present invention can be embodied in a multitude of different ways as defined and covered by the claims.

For convenience, the following description will be organized into the following principal sections: Introduction, System Overview, Operating Features of the Expert System, Software Structure, Data Navigation, Additional System Processes, Knowledge Base, Extended Analysis, Benefits, and Summary.

Introduction

The expert system of the present invention expands upon the state of the art by providing automated analysis to electropherogram data outputted by genetic analysis programs. In the illustrative embodiments to follow, the present invention is configured to accept the output files from ABI's GeneScan and GenoTyper software, however, output from other genetic analysis programs may also be conveniently used. By using the expert system of the present invention, experts will be able to review more cases in less time. Expert analysis will be less expensive and more widely available. Additionally, with the objective analysis performed by the present invention, fewer analysis mistakes will go unnoticed and most importantly, resulting in more reliable and consistent interpretation of DNA evidence. Further, the data is provided in a format that can be easily presented and explained to laymen.

System Overview

An exemplary implementation of the present invention is directed to a computer-implemented method and system for the expert analysis of forensic DNA evidence. While specific hardware and software languages will be referenced, it will be understood that a panoply of different components and software languages could be used to implement the present system.

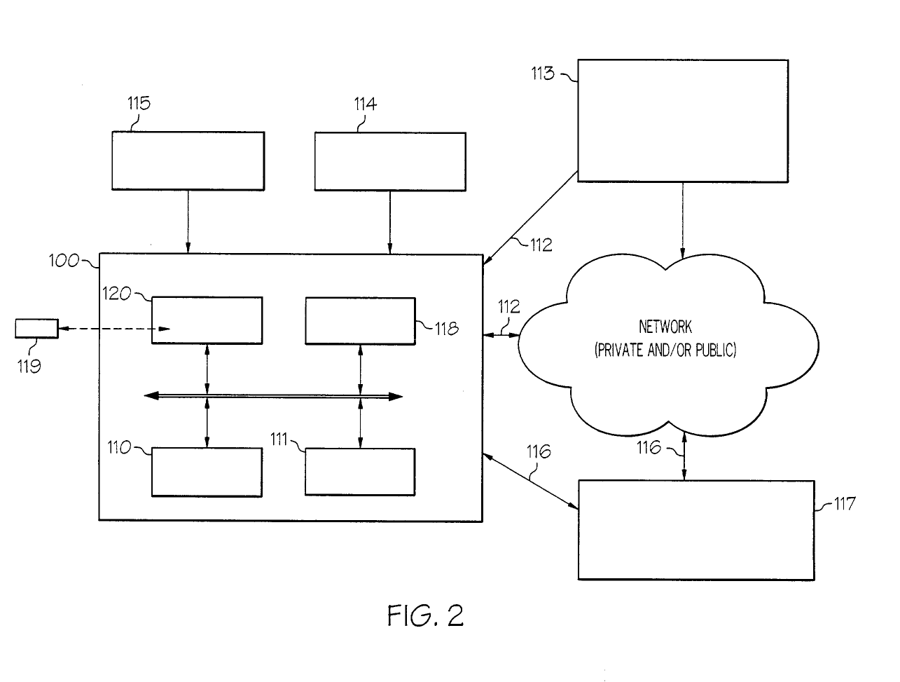

As shown schematically in FIG. 2, an expert system 100 of the present invention includes at least one central processor 110, for example an Intel Pentium™ processor or Motorola Power PC, and a storage device 111, such as a hard disk, flash memory, compact disk, magnetic tape, or any other suitable computer storage medium for storing standard fragment patterns. A communications link 112 is provided such that the expert system 100 receives output from a genetic analyzer 113. The communications link 112 may be a network (private and/or public), landlines, wired, and/or wireless connections.

The output of the genetic analyzer 113 may be either raw DNA detection data or electropherogram data (hereinafter “the received data”), provided in any conventional electronic format. Additionally, the received data raw detection DNA data or processed electropherogram data may be provided on a computer-readable medium 119, such as a flash memory, CD, DVD, floppy, removable hard drive, and read from a computer-readable device 120, such as external flash memory reader, CD reader, DVD, floppy, and USB and Firewire device ports.

The analysis of the expert system 100 may be output from processor 110, in print form using printer 114; on a video display 115; or via another communications link 116 to another computer system 117. Additionally, the analysis from the expert system 100 may be utilized by the processor 110 in other suitable applications, such as for example, presentation and visualization programs, such as Microsoft's Office Suite, web browsers, whiteboard software, and the like.

It is to be appreciated that in the discussion to follow, that for purpose of illustration the programming instructions of the expert system 100 are shown as existing or residing in main memory 118. Persons skilled in the art to which the invention relates understand that programming instructions (software elements) are typically executed from main memory 118 and may be fetched into the main memory on an as-needed basis from other sources, such as, storage device 111, or over a network from another computer, such as computer system 117, and/or other network server/device. As such, persons will appreciate these programming instructions may or may not actually exist simultaneously or in their entirety in main memory 118.

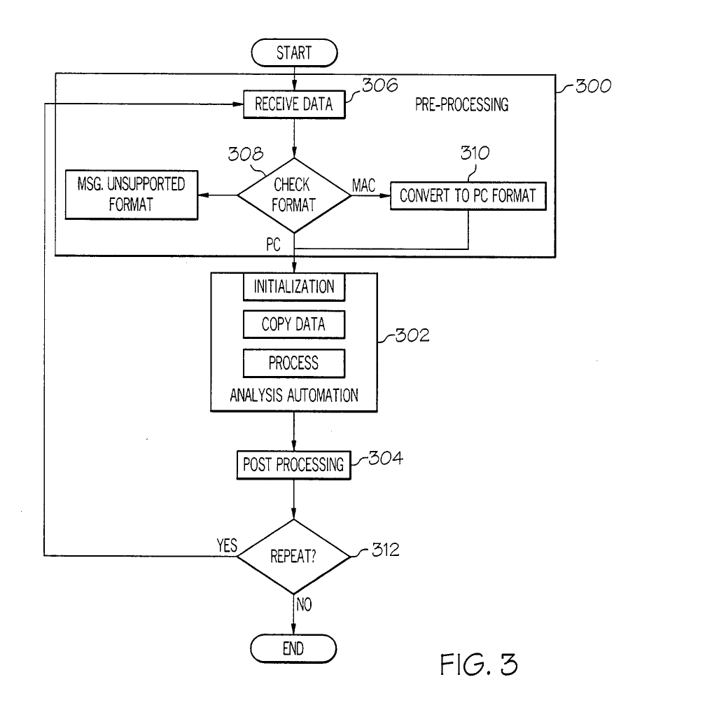

Additionally, a user can configure, initiate, and control the execution of the programming instructions on the expert system 100 in the conventional manner. In addition to the software of the present invention comprising the elements and routines discussed below, the expert system 100 further includes a conventional operating system to facilitate the execution of such programs and other functions typically performed by operating systems. The operating features of the expert system is shown in FIG. 3 and described hereafter.

Operating Features

The expert system 100 of the present invention offers automated analysis with very little setup time. It also offers an easily accessible and novel method for viewing the analysis files. The operating features of the present invention are provided in three phases of execution: a pre-processing routine 300, an automated analysis routine 302, and a post-processing routine 304.

The above-mentioned overarching routines are linked together according to the flowchart illustrated by FIG. 3. In particular, the preprocessing routine 300 prepares the sample files from genetic analysis programs for analysis. The automation analysis routine 302 mirrors exactly how an expert would organize and analyze the received analysis data, and the post-processing routine 304 creates the navigation web page to access the output files in a convenient format.

The software of the expert system 100 performing the three phases of execution includes the following code modules for which source code is included in the attached CD-ROM Appendix:

A. AutoGeneScan.wbt—a winbatch program tied to the front-end GUI, which automates the analysis of the data files from genetic analysis programs.

B. genepp.pl—a Perl module called from AutoGeneScan.wbt to copy a source directory to a computer readable medium, and layout and setup the output files of the expert system.

C. genehtml.pl—a Perl module called from AutoGeneScan.wbt to import the data files from genetic analysis programs.

D. quicklinks_shortname.pl—a Perl module called from AutoGeneScan.wbt to construct a HTML page to aid users in the display and navigation of the PDF output files produced by expert system.

E. Front_End.form & dlgDirSelect.frm—form templates used by a visual basic program to create a front-end Graphic User Interface (GUI) for users and shell for running subsequent routines.

In the current version, automated analysis is performed on an IBM-compatible PC running the Windows operating system. Those skilled in the art, however, recognize that other operating systems may be used. As previously mentioned, the DNA evidence provided to counsel (i.e., defense counsel) is typically on a CD containing the data from a genetic analyzer, which is typically in the Macintosh format. As part of the pre-processing routine 300, the data received by the expert system in step 306 is checked for format in step 308. If the data is in Macintosh format, then in order to analyze such data, the expert system 100 in step 310 converts the Macintosh formatted data to PC formatted data.

To carry out this formatting, the expert system 100 calls in the software module genepp.pl. The genepp.pl module copies the raw data files from a selected directory on a local or remote system or on a computer-readable medium (e.g., Zip Disk, flash memory, CD, DVD, etc.), and renames the files such that it has the proper extension and read/write attributes. File creation and modification dates are also stored to ensure that the data has not been modified prior to being admitted into evidence. If necessary and available, the expert system 100 can also run for the user in the pre-processing routine 300, any needed analysis program, such as ABI's GenScan and GenoTyper programs on the received data and save the output data to a .pdf file.

After pre-processing routine 300, the received data will have a .fsa extension and along with the predetermined read/write permissions. The directory file containing these modified files will also contain three text files indicating the directory creation date, the directory modification date, and the directory last accessed data. It is to be appreciated that these text files contain the timestamp information for the files in the original run directory on the provided computer readable medium as evidence, along with the size standard and analysis parameters used for each analysis run.

Next, or if pre-processing is unnecessary as determined in step 308, the automated analysis routine 302 is performed by running the software module AutoGenScan.wbt, which is a WinBatch program. As those skilled in the art are aware, WinBatch includes the Windows Interface Language (WIL), which is a fully functional language to operate Windows. Commands include mouse clicks and keystrokes, as well as more complicated Windows functions such as file operations, registry editing, and dialog box creation. As such, the AutoGenScan.wbt module includes code for every mouse click and keystroke of an expert performing the analysis task according to standardized forensic procedures. Actions are performed at the correct time and order by implemented timers, such that processing finishes or actions cannot be performed when the program cannot register them.

In the current implementation, the expert system 100 is programmed to look at the windows opened as a cue to begin processing. For example, when the print dialog has closed, the expert system 100 is programmed to know that the current analysis output screen has completed printing, and thus can then continue with the next step in the analysis process.

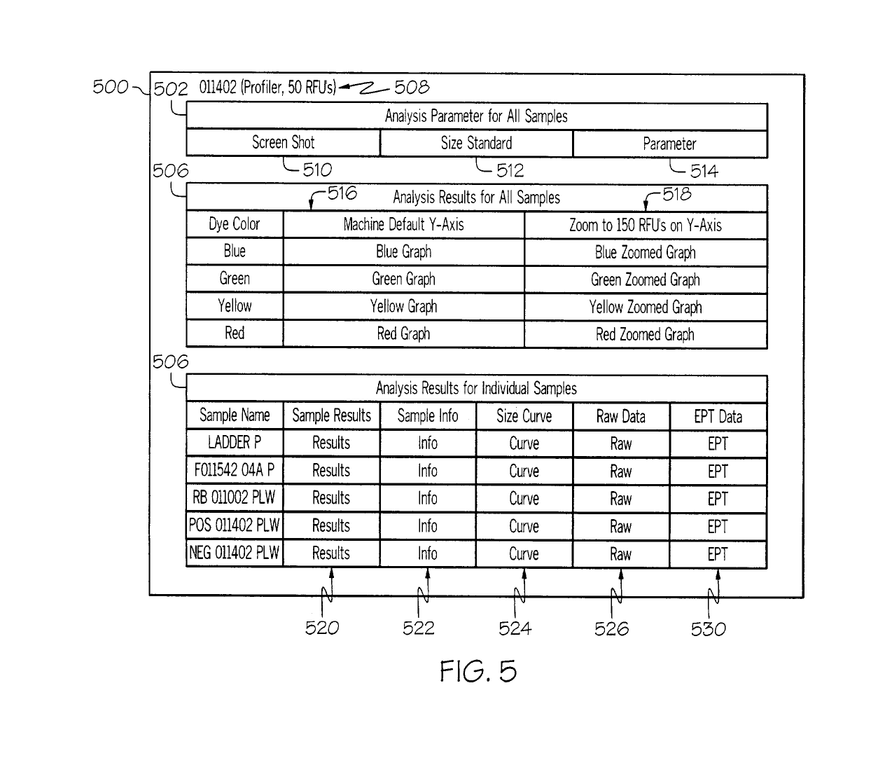

Once the automated analysis routine 302 has finished, the post-processing routine 304 is performed by calling the software module quicklinks_shortname.pl. The quicklinks_shortname.pl module creates an hypertext-markup language (“HTML”) formatted navigation web page 500 (FIG. 5) which allows easy access to all of the analysis files. Since the HTML format is universally supported, navigator web page 500 and the output files of the present invention can be viewed on any computer with a web browser and Adobe Acrobat Reader.

The software module quicklinks_shortname.pl works by looking through the received data and organizing included data files by run name, analysis type (Profiler Plus or Cofiler), and cutoff threshold (50 or 150 RFUs). HTML navigation buttons are then created for each output file, which are provided on the navigation web page 500 in organized tables, which are described hereafter in the next section. If there is no further data, which is checked in step 312, the execution or runtime of the expert system 100 ends.

Software Structure

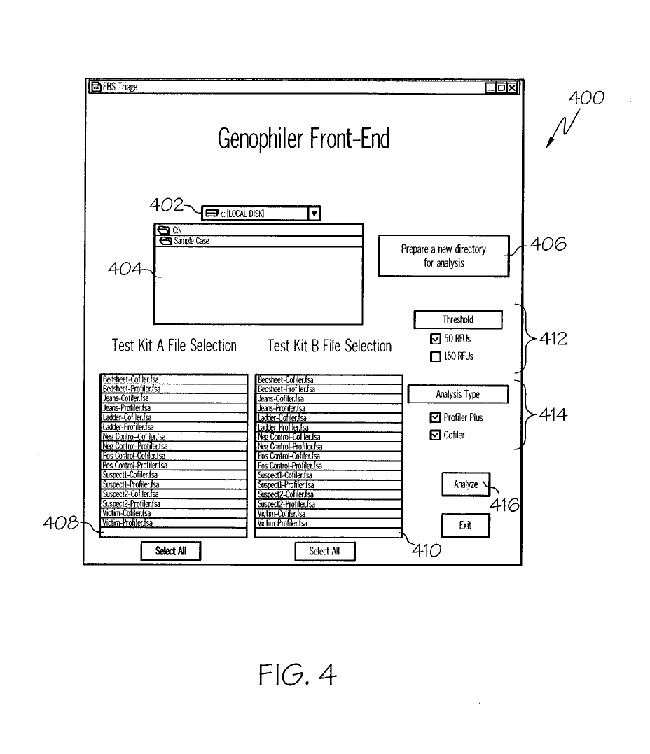

The routines of the expert system are tied together with a front-end graphical user interface (GUI) 400, which is illustrated by FIG. 4. The GUI 400 provides all of the options needed for a user to set up the automated analysis of the received forensic DNA data from a single screen. As illustrated, the user may select the directory containing the sample files for analysis from the provided path drop down box 402, and path selection box 404. Additionally, the user can prepare a new directory for the resulting data of the expert system by selecting on the illustrated “Prepare a new directory for analysis” button 406. Selecting button 406 opens a window for the user to indicate/create the path for the resulting analysis files.

To select the files for automatic analysis by the expert system 100, file selection boxes 408 and 410 are provided. It is to be appreciated that testing for the 13 different genetic loci in the FBI's CODIS database currently requires the use of two STR test kits. In each file selections box 408 or 410, the user may select which files are to be processed by expert system 100.

Additionally, from GUI 400 the user can select parameters, such as the threshold level

RFU or 150 RFU) and type of analysis, with check boxes 412 and 414, respectively. The illustrated options available for selection via analysis type check box 414 are Perkin Elmer's Profiler, Cofiler, or both for analysis. Although, the current standards in testing kits are selectable, it is to be appreciated that other STR testing kits may also be listed on the GUI and selected for analysis by the expert system 100 of the present invention.

Once the data files and parameters have been selected, the user only needs to click on the “Analyze” button 416 and the analysis is automatically performed. The analysis of the expert system 100 can be performed unattended, with multiple analysis runs if desired. At the completion of the analysis, output from the expert system is then stored in Adobe Acrobat PDF format in the user designated directory, in the following directory format:

. . . /Analysis/<Run Name>/<Cofiler/Profiler>/<50/150 RFU Cutoff>/<Sample Name>

The user can access the analysis files using the navigation web page 500 on any PC having a web browser. Additionally, the user can access the files directly from the designated directory. A discussion on the navigation web page 500 now follows with reference made to FIG. 5.

Data Navigation

The expert system's navigation web page 500 is broken into three tables for each run, which are respectively designated by the following table labels: “Analysis Parameters for All Samples” 502, “Analysis Results for All Samples” 504, and “Analysis results for Individual Samples” 506. Each table of navigation buttons (links) enables the user to easily access and view the receieved electropherogram data including all of the analysis parameters and combined and individual results for each DNA sample run. Each run is identified on the navigation web page by a header label 508, which displays the run name, analysis type (Profiler or Cofiler), and the RFU cutoff value (50 or 150 RFUs). A discussion of each table now follows.

The first table of navigation buttons (or links) 502 provides a convenient reference structure for quickly retrieving and viewing the analysis parameters used for all samples by conventional genetic analyzers, such as, for example ABI's GeneScan. As illustrated, the buttons include a “Screen Shot” 510, “Size Standard” 512, and “Parameter” 514. The “Screen Shot” button 510 when selected opens a window which displays what samples were analyzed and what parameter files were used for the run. The “Size Standard” button 512 when selected opens a window which displays the size standard file (e.g., the “>GS-500 ALL” parameter screen). The “Parameters” button 514 when selected opens a window which displays all of the size information for the run, including RFU cutoff and smoothing options.

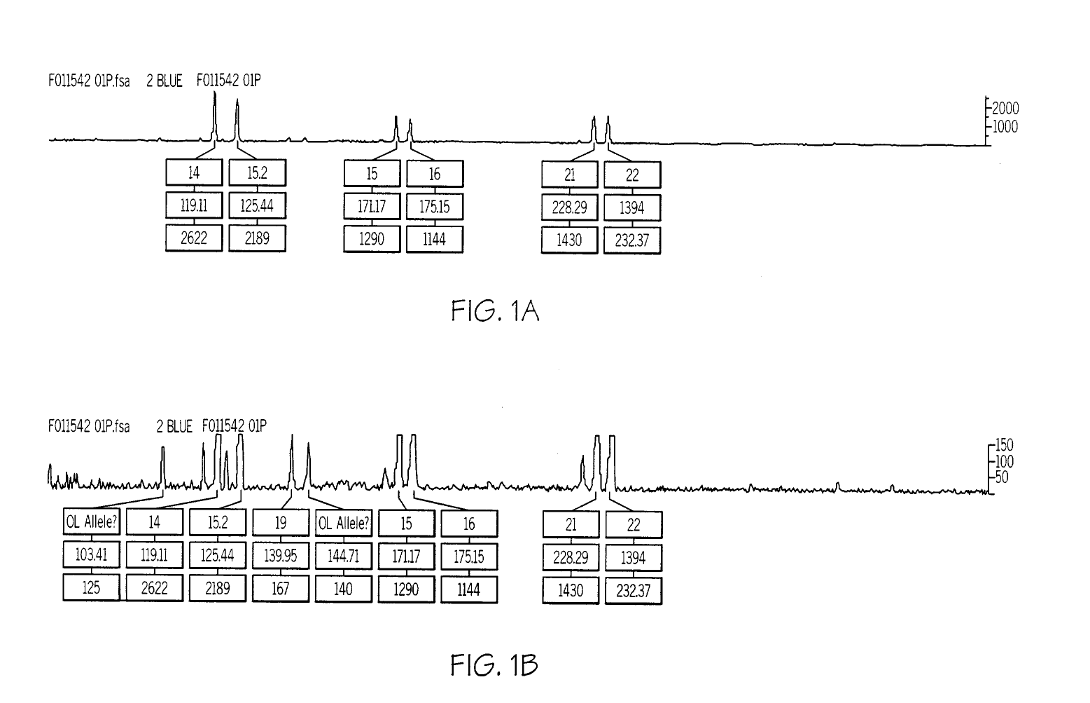

The second table of navigation buttons (or links) 504 provides a convenient reference structure for quickly retrieving and viewing the electropherogram outputted by conventional analysis programs, such as for example, ABI's GeneTyper. As illustrated, when any of the first column buttons 516 are selected, a window displays the machine default y-axis graph (e.g., FIG. 1A) for the corresponding dye color (blue, green, yellow, red), which allows a user to quickly view different locations of DNA. The second column of buttons 518 provides a zoomed electropherogram (FIG. 1B) that enable the user to analyze the y-axis graphs for the corresponding dye colors more closely. Such a zoom feature is useful to find small peaks and artifacts that may be hidden when using the machine's default graph settings. Additionally, with the zoomed graphs (e.g., FIG. 1B), the user is able to closely examine smaller peaks and artifacts to see whether they are alleles from a possible secondary contributor(s) that may have been unnoticed and/or unreported by a testing laboratory.

In the illustrated example shown in FIG. 1B, the expert system 100 analyzed the same raw data provided as evidence but produced an electropherogram having additional allele labels than the testing laboratory's graph (FIG. 1A). In this example, the additional allele labels resulted from re-run operator decisions made in the original analysis of the raw data generated from the genetic analyzers. Inspection of the “zoomed” electropherograms using a setting of 150 RFU's revealed the presence of alleles that a conventional genetic allele typing program, such as ABI's GenoTyper program, determined met the criteria that made them appear to be true alleles. These additional alleles could have been discarded from the testing laboratory's electropherogram (FIG. 1A) for a number of reasons, including the determination that they were artifacts or noise. Determinations such as these can be very subjective and arbitrary making it essential that they be drawn to the attention of an expert for additional review. Accordingly, the expert system of the present invention flags such additional allele labels for later inspection by an expert.

The third table of navigation buttons 506 provides a convenient reference structure for quickly retrieving and viewing the analysis results of individual samples outputted by conventional analysis programs. Using the third set of navigation buttons, the user is able to quickly see the machine settings used to analyze the samples in order to quickly assess whether any possible problems exist. It is to be appreciated that all of the individual sample information is organized in such a fashion that the user can see all of the peaks present in the electropherogram and their attributes. Therefore, the user can examine the height and area of smaller unlabeled peaks in the output of the genetic analysis programs.

As illustrated, the columns of buttons include “Results” buttons 520, “Info” buttons 522, “Curve” buttons 524, “Raw” buttons 526, and “EPT” buttons 528 for each sample in the run designated by the header label 508. The “Result” button 520 when selected for a particular sample opens a window which shows the unlabelled electropherograms. The “Info” button 522 when selected for the particular sample opens a window which shows the setting of the conventional genetic analyzer, such as an ABI's 310 Genetic Analyzer. The “Curve” button 524 when selected for the particular sample opens a window which shows how well the sample matched the size standard.

The “Raw” button 526 when selected for a particular sample opens a window which shows the unfiltered data from the genetic analyzer. The “EPT” button 530 when selected for a particular sample opens a window which shows the voltage current, laser power, and temperature readings of the genetic analyzer during the run on the particular sample. In this fashion, any problems with the actual analysis of each sample can be quickly identified using these tables of navigation buttons. Additional system processes may be provided to the expert system 100 to automate the identification and classification of other potential problems in the received data, which are discussed hereafter.

Additional System Processes

The expert system of the present invention may be provided with a number of routines used to make classifications on the received STR DNA data. It is to be appreciated that conventional genetic analysis programs currently only provide to the user in a report the time at which the peak was seen (its position on the x-axis), its peak height, and its peak area. Additionally, conventional genetic typing programs only filter out peaks that are thought to be erroneous and labels true peaks with their number of repeats. The expert system of the present invention, however, can expand upon the state of the art by making characterizations of the received electropherogram data based on the few attributes provided by conventional genetic analysis programs. The results of these characterizations may then be used as a guideline to flag possible problems that can be examined by a DNA expert later.

The characterizations of the expert system are executed by programming the processor 110 to simulate the reasoning of human experts in the particular problem domain of triaging forensic DNA. The programming instructions include productions (a set of programming if-then rules), and an inference engine, which is programming which uses the production to reason through a given inputted data set in order to produce a desired output. The firing of a rule causes an action to be taken; e.g., flagging a potential problem. In particular, the programming instructions are used to determine whether the received data on the DNA sample has any glaring problems. The programming instructions may be permanent, as in the case where the processor 110 has a dedicated EEPROM, or it may be transient, in which case the programming instructions are loaded from the storage device 111 and run from main memory 118.

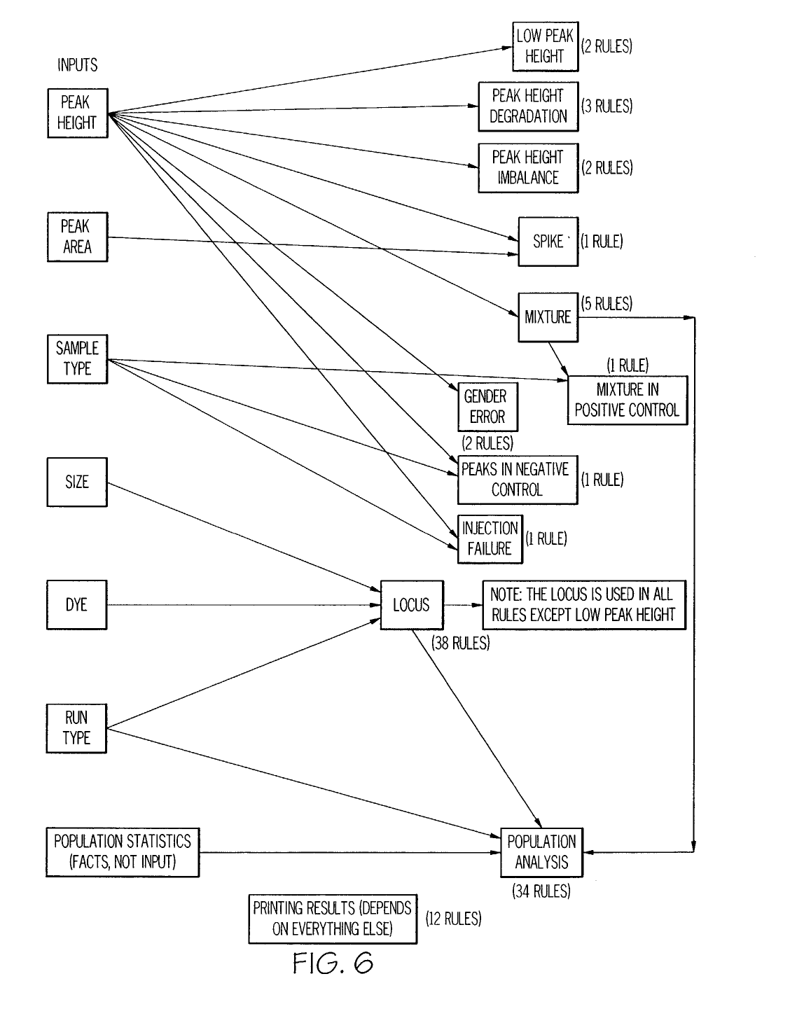

After initialization and loading of data, a list of rules is scanned repeatedly until inference is complete, and then the results are output. Sample input and the corresponding results are shown in FIG. 6, which is a rule map for the expert system. As mentioned previously, the received data on a sample from a conventional genetic analyzer includes peak height, peak area, sample type, size, dye, and run type. FIG. 6 shows rule map for the expert system and the principal results inferred from the received data: low peak height, peak height degradation, peak height imbalance, spike, mixture, mixture in positive control, peaks in negative control, gender error, injection failure, locus naming, and population analysis. A discussion on the rules used by the expert system follows hereafter.

In one particular embodiment, the expert system comprises a knowledge base and an inference engine. The knowledge base comprises facts and rules. Facts comprise the received data or any other data input, such as population statistics, and of anything inferred from this input by the expert system. Such inferred facts may include: the recognition that some alleles have unusually low peak height; peak heights that are indicative of degradation; peak heights that are imbalanced and indicative of mixtures; spikes; unaccounted alleles in positive control; peaks in negative control; gender typing errors; injection failures; locus naming; and population analysis, or anything else not explicitly stated in the initial rules.

Typically, rules have the form “if certain conditions are met, then take certain actions (or draw certain conclusions).” For example, a rule could be implemented which checks for possible peak height imbalance between two alleles by the following statements:

(def rule imbalance01

(DNA (name ?allele1) (locus ?locus) (p_height ?pheight1))
(DNA (name ?allele2&˜?allele1) (locus ?locus)

(p height ?pheight2))

(RESULTS (name ?allele1) (peak_height_imbalance nil))
(RESULTS (name ?allele2) (peak_height_imbalance nil))
(test (>= ?pheight2 ?pheight1))
(test (<= ?pheight1 (* ?pheight2 .70)))

?f 1 <− (RESULTS (name ?allele1))
?f 2 <− (RESULTS (name ?allele2))

=>
(modify ?f1 (peak_height...imbalance POSSIBLE))
(modify ?f 2 (peak_height....imbalance POSSIBLE))

)

This rule says to find an allele within a locus, and remember its peak height. Then find another allele in the same locus, with a different name, and remember its peak height. Find the results field for each of these alleles, and then test to see if the peak heights differ by more than 30%. If so, it is possible that a peak height imbalance exists. Note that this rule has an implied criterion that the locus must be heterozygous, thus two different peaks must be found in order to be compared.

The “sample type” label only needs to be used if the sample is one of the control groups. These include the positive control, negative control, and ROX size standard samples. The locus label is necessary and should contain the actual locus name corresponding to the current analysis sample's dye and size. The locus can be found by looking at the placement of the allele on the output graph of the conventional genetic analysis program, such as ABI's GeneType program, or by utilizing a software tool to do preprocessing.

It is also possible to have “else” clauses in the rule. The rules can be embedded in the program, executed from a database, or read from a file. The inference engine is a general mechanism to infer new facts from data using the rules. The inference engine can use meta-rules (rules about rules) to guide the reasoning process; e.g., rules about what ordinary rule to apply next, or about how to resolve conflicts. For each rule, meta-rules may be used to determine whether a rule should be permitted to fire.

For example, a meta-rule may be a condition statement that is evaluated for its truth value. This condition statement could be a first rule, wherein if the result of the first rule is true, then the action statement, which may be another rule, can then be executed. Meta-rules are general principles to direct the reasoning of the inference engine. Conflicts occur when the condition statements of two rules are satisfied during a given inference cycle, especially when the corresponding action statements are contradictory. In the preferred embodiment, more than one rule can fire in a given cycle, and rule order and conflict resolution are handled by rule context and rule accuracy. Other conflict-resolution strategies than the one detailed here include use of time tags, rule priorities, or most specific rule first.

Referring back to FIG. 3, the start-to-finish operation of the expert system 100 comprises of loading the electrophoretic data 302, formatting the data if necessary 304, analyzing the data 310, and outputting the results 312. Before performing the hereafter described classifications on the received data, during the analysis automation step 310 counters are initialized, data variables are initialed to “empty”, and the rules are loaded and sorted in main memory 118. Each rule is defined by strings that specify a context, a condition and an action. The context string is simply a tag for the rule. The condition and action strings, however, must be converted to executable form, which can be function calls, equations, or strings that are evaluated by a command interpreter.

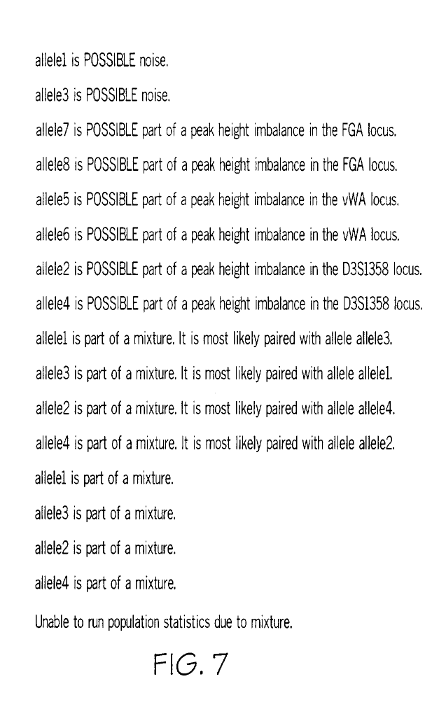

Next, at step 314 raw or processed electrophoretic data is read from a file, or is obtained in real-time. At step 316, the list of rules is scanned one at a time by the inference engine. This scanning process is repeated until no rule fires, or a rule fires indicating that the inference is complete, or a non-recoverable error occurs. At step 312, the results are output, and of which an example is illustrated by FIG. 7, which include but are not limited to indicating “possible” and “probable” problems corresponding to particle alleles. Such labels of “possible” and “probable” can act as a guide to a DNA expert on how much additional analysis is needed after the expert system has been run.

The rules in the illustrated embodiment are all used for forward chaining. This means that the program reasons forward from facts to conclusions. However, backward chaining may be added, which assumes a conclusion and searches for facts to support it. In addition, one or more mechanisms could be employed to handle uncertainty. For example, it would be possible to merge fuzzy logic into the system for this purpose.

In evaluating a fuzzy version of the statement “peak is small”, every peak is determined to be “small” to some degree, but large peaks possess little of this attribute. By avoiding sharp thresholds, fuzzy logic reduces the number of rules required. Additional methods of managing uncertainty that might be employed are certainty factors and probabilities. A discussion on the knowledge base contains the rule sets for classifying low peak height, peak height degradation, peak height imbalance, spikes, mixtures, mixture in the positive control, peaks in the negative control, gender error, injection failure, locus naming, and population analysis now follows.

The Knowledge Base

As mentioned previously, the received electropherogram data generated from the DNA samples are described in terms of alleles present, and each is characterized by peak height, peak area, locus, type of sample, and an arbitrary name. To classify the alleles, the expert system is programmed with sets of rules composed of individual rules. The rule sets and coding for making the hereafter discussed classifications are provided in the Appendix, wherein each rules set looks at an important aspect of the DNA sample. As illustrated by FIG. 6, all of the rules, with the exception of the population analysis, are independent and can be evaluated in any order.

A. Locus Naming

The first rule set of 38 individual rules performs the locus naming and peak counting. For each allele in the received data as input, the locus is determined by looking at the dye name, run type, and size. The peaks in each locus are counted as they are labeled, for use by other rule sets, such as determination of degradation.

B. Determining Mixtures

The second rule set flags alleles as being part of a mixture if three or more alleles exist in a single locus. A common problem in DNA analysis is the presence of a mixture. This occurs when two or more people have contributed DNA to a single sample. The expert system checks the number of peaks present in each locus. If more than 2 alleles are found to be present for any loci, then the entire sample is designated as a possibly mixed sample.

C. Checking for Low Peak Height

The third rule set of two rules looks at the peak height individual alleles. If an allele has a peak height of less than 50 RFUs, it is labeled as being likely to be associated with noise and not signal by the expert system. If an allele has a peak height of less than 150 RFUs, but is greater than 50 RFUs, then the allele is labeled as possible noise but requiring further consideration by an expert since most alleles have a much higher peak.

D. Checking for Negative Peaks in Negative Control Sample

The fourth rule set looks at the negative control sample. The negative control is a blank DNA sample used for comparison purposes. No peaks therefore should exist for the negative control in the received data. The presence of peaks in a negative control is strongly indicative of problems in the sample and subsequent analysis. The expert system is programmed to check whether peaks are present within the negative control. Should a peak be present in the received data, the expert system will flag the peak and alert the user to a negative control problem.

E. Checking for Peak Height Imbalances

The fifth rule set checks for peak height imbalance. If two alleles are found to be associated with a single locus, they should have similar peak heights if they came from the same person. A peak height imbalance is generally considered to occur when one peak is more than 30% higher than another peak in the same locus. Since a sample from a single person contains peak heights that should be roughly equivalent, peak height imbalances are another indication of a sample being a mixture of material from two or more individuals. The expert system is programmed to identify such peak height imbalances in each locus by labeling the left peak X and the right peak Y at a locus. If Peak X<=Peak Y*0.70 then the locus is flagged as a mixture. If any locus in the received data is flagged as a mixture, the expert system will report to the user the locus and which alleles are part of the peak height imbalance such that the user may later analyze the locus and allele to confirm the mixture and de-evolve it if necessary.

F. Checking for Positive Control Problems

The sixth rule set determines whether the positive control contains a mixture. The positive control contains DNA from a single human origin with a characteristic known DNA profile that is used during the analysis to ensure that amplification and typing have been performed correctly. The positive control is used to detect equipment and/or analysis problems such as those associated with contamination. As illustrated by FIG. 6, this rule set relies on the results of the second rule set to determine whether a mixture exists at a particular locus. Accordingly, the expert system 100 is programmed to check that no locus in the positive control contains more than two peaks. Should more than two peaks be present in the received data, the expert system will flag the peaks and alert the user to a positive control problem.

G. Determining Spikes

The seventh rule set checks for a possible technical artifact known as a spike by comparing alleles of the same size observed in two or more different dye colors. Spikes are anomalies that can occur on the analysis output graph. They may appear to be valid alleles, but they have been caused by a power surge during analysis or the presence of dirt in the sample or sensory equipment. If they have the same molecular sizes, one allele or the other may be a spike. Since the expert system does not know which one is the spike, both alleles are flagged for further investigation by the user.

H. Determining Gender Errors

The eighth rule set of two rules checks for two problems with respect to the gender of the sample. The green dye is used to reveal the sex of the sample in the Amelogenin locus. This single locus can often reveal problems if they exist in the sample. Possible alleles for this locus are either X, meaning a woman (XX), or an X and Y, meaning a man (XY) was a contributor to the sample. If a Y allele is found but no X is detected, the sample is flagged. Also, if three alleles are found in the Amelogenin locus, the alleles are also flagged with a gender error.

I. Resolving Mixtures

The ninth rule set of four rules performs a more detailed analysis of the results from the second rule set of determining mixtures. Given that it has already been determined that a mixture exists in the second rule set, it is useful to determine not only where the mixture exists, but which peaks might possibly be from the same sample. This rule set considers only the simplest possible mixture cases where only two individuals have contributed to a sample. If four peaks exist, the rules attempt to cluster them together into two pairs based on peak height. If three peaks exist, the rules attempt to cluster together the two that are most likely to come from the same sample. Additional analysis routines may be performed by the expert system to further resolve mixtures, which is discussed hereafter in a later section.

J. Determining Sample Degradation

The tenth rule set of three rules flags possible degradation if two alleles exist in the same locus and the peak height of allele X is greater than the peak height of allele Y. Samples can also degrade during analysis, and this is visible on the electropherogram with progressively smaller peak heights (and areas) as allele sizes increase. To check for degradation when the dye contains both heterozygotes and homozygotes, the two peaks of a heterozygote are added together to produce a single peak, which can then be compared to homozygotes, or heterozygotes which have received similar treatment. Additional analysis routines may be performed by the expert system to further determine degradation, which is discussed hereafter in a later section.

K. Checking for Injection Failure

The eleventh rule set checks for a possible injection failure by comparing the peak heights in the ROX sample. The ROX sample corresponds to a set of DNA fragments of known size that is used as a size standard. Its characteristics are known, and it is used to determine and compare the sizes of the unknown samples. Each allele in the ROX sample is expected to have the same peak height. If they are not within 10% of each other, the sample is flagged.

L. Assigning Racial Probabilities

The twelfth rule set uses the data from the locus determination and user input to assign probabilities of racial derivation to the input sample. This is only attempted if a mixture does not exist. Based on empirical data collected by the FBI and other agencies, one can use the probability of a given locus existing in a racial group to determine the probability that a given sample came from that group, based on all of the alleles present.

M. Printing Results

The final rule set is simply for printing purposes. Each of the above 12 rule sets has a salience value lower than 0, and they are given progressively lower salience values so that the output is always printed in the same order. This standardizes the output.

Extended Analysis

As mentioned previously, additional processes may be preformed by the expert system to further classify the received data. In particular, the following optional automated analysis may be performed by other embodiments of the present invention.

A. Classifying Peaks

When evaluating a DNA sample, it is important to know whether the received data is satisfactory. DNA analysis is a sensitive process that relies on the fluorescence of dyes due to excitement by a laser through a small capillary tube. As pieces of DNA float through the capillary tube and pass the laser, their presence and size is recorded on an electropherogram graph. However, the presence of other materials will also cause the reflectance of light and the presence of peaks that are not DNA. Air bubbles, urea crystals, dye blobs, voltage spikes, and sample contamination can cause the output graph to report a false peak. Therefore, classifying peaks can separate real peaks from other anomalies that may be misinterpreted as actual DNA data. The expert system classifies single peaks based on peak height and peak area. The expert system 100 discerns between what is likely to be a true peak and what is likely to have arisen due to a technical artifact by using Equation (a):

Peak Area>(−2.1*Peak Height)+3600−>Good Peak (a)

The equation was derived from observations made on verified examples of true and false DNA alleles (an allele is one version of an STR locus that is present in the sample). The observation data set was created using five cases, consisting of 36 samples totaling 779 individual peaks that included 571 true and 208 false alleles.

A feature vector was created for each single peak in the data set which included the case name, analysis kit type (Profiler Plus or Cofiler), the RFU cutoff threshold, the sample name, the dye color, the sample peak number, time the peak was observed, size of the peak, peak height, peak area, data point, name of the locus in which it resides, and the number of repeats. Samples with an RFU cutoff value of 50 were used to ensure that the maximum variety of peaks were present in the data set. A scatter plot of the set of good peaks indicated that the data points appeared to follow a fairly linear trend. Plotting the bad peaks along with the good peaks resulted a disguisable decision boundary, which resulted in Equation (a).

Running Equation (a) in application of the expert system 100 on the above noted test data set resulted in seven of the true peaks being classified as “false” as the decision boundary resulting in Equation A did not account for any peaks less than 300 RFUs. In practice, most peaks fall above the decision boundary as the data set showed, but it is possible to have a true peak with far less amplitude.

Additionally, only one of the bad peaks in the data set was classified as a “true” allele, as most false allele peaks remained around 100 RFUs. Accordingly, the total error rate for the test data set was about 1.025%. It is believed that with more data, this value is likely to increase because of the peak height constraint on what is considered a true allele peak. A peak can still be reliable if it is below 300 RFU's, it is just not as likely to appear. However, since it is more desirable to have a false negative than a false positive, the expert system then flags such bad peaks such that an expert may look at them at a later time.

Furthermore, a quick check to the number of peaks will reveal whether the peak classifier has returned too few true allele peaks. For example, a sample from one individual should have three to six peaks. If a sample contains peaks less than 300 RFU's, then it is likely that all of the peaks in a sample will fall below the predetermined decision boundary (Equation (a)), resulting in too few good peaks.

Consequently, no peaks will be classified as being associated with a true allele, and the user will have to resort to analyzing the sample by hand. In either situation, it is to be appreciated that the expert system 100 does not replace the DNA expert, it only acts as a tool and guide to highlighting possible problems, thereby permitting the experts to complete the analysis in a more efficient and organized manner.

B. Matching Samples

The second classification of the expert system is to match two different samples and return their level of similarity. The number of repeats in a peak is the key form of identification of a sample. If all of the suspect's peaks determined with a conventional STR-PCR typing system are present in a sample, then it is very likely that they contributed to the sample. However, if even one peak is missing, an argument can be made that the suspect can be ruled out as a possible contributor. Although, the process of matching samples is simple, it does require time to find two corresponding samples in the output by eye, record their repeats, and perform a match. Accordingly, the purpose of this classification is to automatically flag those samples that have any degree of similarity so that they may be later examined by an expert.

It is to be appreciated that labs will typically provide the DNA sample and the suspects DNA data in a tabular format, such as for example, like in Table 1:

TABLE 1

| | Locus 1 | Locus 2 | Locus 3 |
|---|---|---|---|
| DNA sample | 13, 14, 17, 18 | 15, 16, 18 | 20, 24 |
| Suspect | 13, 17 | 15, 16 | 20, 24 |

Accordingly, the expert system compares all of the peaks in the sample with the suspects to find a correspondence. If both samples are the same, then it is likely that the two samples came from the same person. If one sample contains all of the peaks from another sample, then a second or third person's peaks will be the ones remaining. Resolving problems occur when two people have many of the same peaks in common. It is often necessary in this case to look at other loci to get a better idea of who contributed to the sample and the level of similarity.

The expert system is programmed to compare two DNA samples and return their level of similarity along with the peaks that both samples have in common by using Equation (b):

[common, match]=DNAmatch(sample 1, sample 2) (b)

where the function DNA match operates on the samples provided in a two-dimensional array. The first row of the array contains the number of repeats for each peak, and the second row of the array contains the locus number (1-4) to which the peak corresponds. Since two samples from the same dye are being compared, precise locus names are optional.

The DNAmatch function returns two objects. The first is an array called common, which contains all of the peaks that are in common in both samples. The second returned value is match, which details the level of similarity between the samples. The values of match can be no match, a partial match, a perfect match, sample 1 is a subset of sample 2, or sample 2 is a subset of sample 1. Partial matches rule out the presence of a sample because all peaks must be present in both samples to consider inclusion.

C. Resolving Mixtures

The third classification is to identify and resolve mixtures of two or more people in a single sample. When more than one person contributes to a sample, the sample is classified as a mixture and is often difficult to resolve. The number of matches increases greatly when multiple people contribute to a sample because any combination of peaks may be present. Accordingly, the purpose of this classification is to flag when a mixture exists and to identify what the most likely set of peaks will be for each contributor.

If more than one person contributes to a sample, then more than two peaks will be visible at any locus. However, it is known that in a mixture, peaks of similar height are most likely to be from the same person because the amount of DNA present in a sample is usually constant for each contributor. It is also known that DNA belonging to each individual in the samples often has a different concentration and hence different peak heights.

The typical values for which the peak heights can be considered similar to each other are when the ratio of the lower peak to that of the higher peak is greater than 0.7 (Dmerge). Conversely, it can be said that two peaks are dissimilar if the above ratio is less than 0.3 (Dsplit). Accordingly, the expert system may utilize pattern recognition techniques (e.g., k-means, isodata, fuzzy k-means) to cluster the sample data based on peak height in order to separate contributors. In particular, the clustering algorithm of the expert system is based on the following observations in a typically sample:

There is no prior knowledge as to how many sources contributed to the mixture, but that the approximate number of sources can be estimated by observing the peaks that differ grossly.

Two peak heights from different sources will differ in all likelihood in their values by a significant value.

The datasets are not going to be extremely large. For one dye color used, the expected dataset number in the region of 10-20.

Based on the above observations, in one embodiment, the ISODATA function is believed to provide adequate screening results by forming a separate cluster for the peaks belonging to each individual that forms a part of the mixture. In other embodiments, other pattern recognition algorithms, which separate contributors' STR DNA by resolving mixtures, may be used. The implementation of the ISODATA function, though simplistic in its assumption and implementation, is a very powerful tool that can be used for resolving mixtures. The symbols used by the ISODATA function are listed in Table 2.

TABLE 2

| | |
|---|---|
| Nd | expected number of clusters |
| Dsplit | minimum distance to split clusters (in this case ratio of the low |
| | peak to high peak being less than 0.3). |
| Dmerge | maximum distance to merge clusters (in this case ratio of the low |
| | peak to high peak being greater than 0.7). |
| Nc | number of clusters for k-means algorithm |
| mu | mean vector to be used by k-means |
| lbl | current labels of the clusters |
| Lc | labels of the clusters in last iteration of k-means |
| Data | data that needs to be clustered |

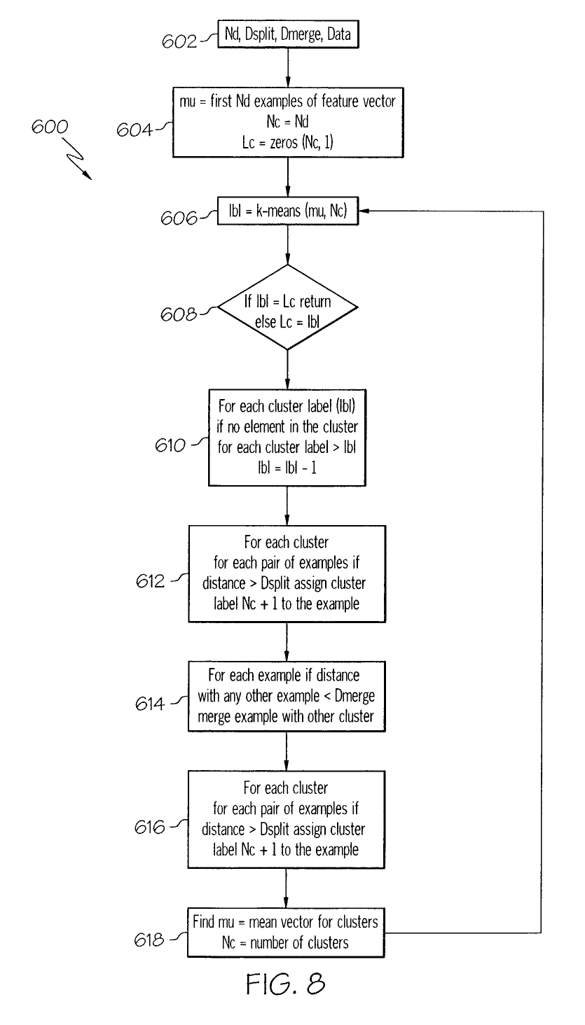

A flow chart of the ISODATA function 600 for the present invention is illustrated by FIG. 8. As illustrated, in addition to reading the data and the default values for Dsplit and Dmerge, the expert system 100 requests the user to input the expected number of clusters Nd in step 602. The Dsplit and Dmerge value may be re-assigned by the user, if desired. In step 604, the first Nd example values of the feature vector (explained previously above) are chosen as the mean vector mu for the k-means clustering, and sets the number of clusters Nc required to Nd. The cluster label of the last iteration of k-means Lc is also set to zero. Since k-means might create clusters, which have no elements, empty clusters are eliminated. In step 606, the current label of the cluster lbl is set equal to the closest cluster determined by k-means. In step 608, cluster label Lc is set equal to the current label. In step 610, each of these clusters is then split into one extra cluster each for the clusters that are one or more elements apart.

All the examples are then compared to all the other examples to check whether a cluster exists with which any of the examples can be merged in step 612. If a cluster is found, then the example is merged with the other cluster in step 614. As a result, there might be elimination of some clusters. Accordingly, in step 616 the redundant cluster labels are removed. In step 618, the means and the number of clusters for the final cluster set are found, and the next iteration of k-means is initiated with these values. The algorithm is considered converged in step 608 when the k-means algorithm does not create a change in the cluster labels. All mixtures returning at least two clusters are then flagged by the expert system as a possible mixture, which can be later analyzed by the user in order to separate each contributor's alleles found in a cluster.

D. Classifying Spikes (EPT Data Problems)

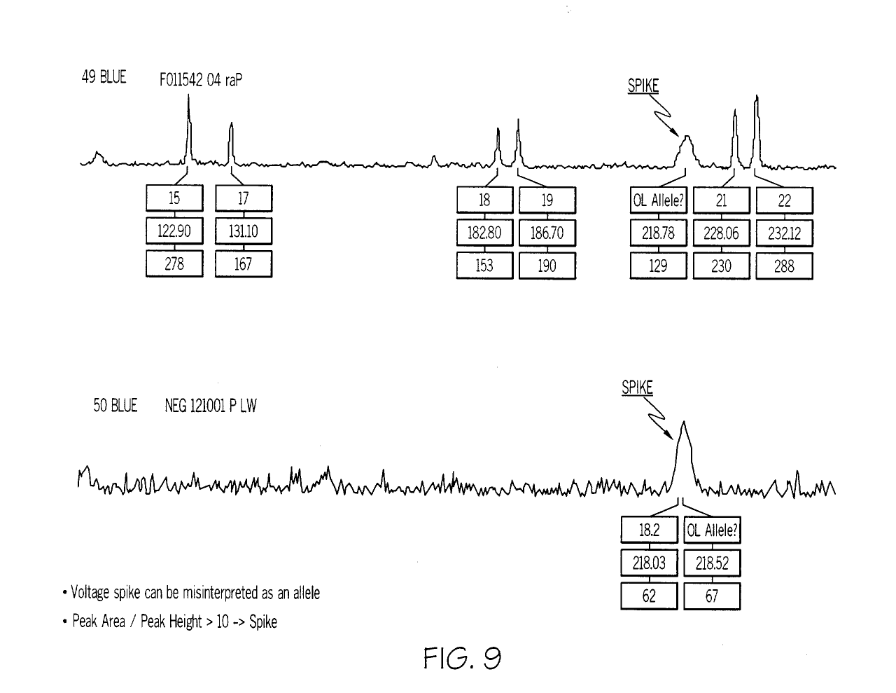

The fourth classification is to identify spikes in a single sample. In some instances, a voltage spike can create what appears to be a valid peak as illustrated by FIG. 9. Additionally, the spike's large height and area may cause it to fall on the good side of the decision boundary of the classifying peaks equation (a). Accordingly, the expert system 100 is programmed to determine when a spike occurs such that a user does not have to study every true allele peak in the received data.

From analyzing the true allele peaks in the above-mentioned data set, it has been observed that the ratio between peak area and peak height is almost linear. Even though spikes can have a large peak height, most of them fall below 500 RFU's. It has also been observed that there exists a large difference in the variance between the ratio of the good peaks and the bad. All of the true allele peaks fall below the ratio of 10. Examining the false peaks with a ratio greater than 10, it has been observed that almost all of them could be classified as spikes. Accordingly, the expert system 100 is programmed to use this ratio value as a decision boundary for classifying spikes in the received data, which is defined by Equation (c):

Peak Area/Peak Height>10−>Spike (c)

A more conservative estimate would be to check if the ratio was greater than 12 or 13, but 10 is sufficient for providing the needed indication of a possible problem in the received for later analysis by an expert. Such flagged peaks or power level problems can also be currently determined by looking at the EPT data generated by conventional genetic analysis programs which should show constant power levels for all sample runs, and power spikes matching up with the flagged peak to confirm a bad peak.

Degradation

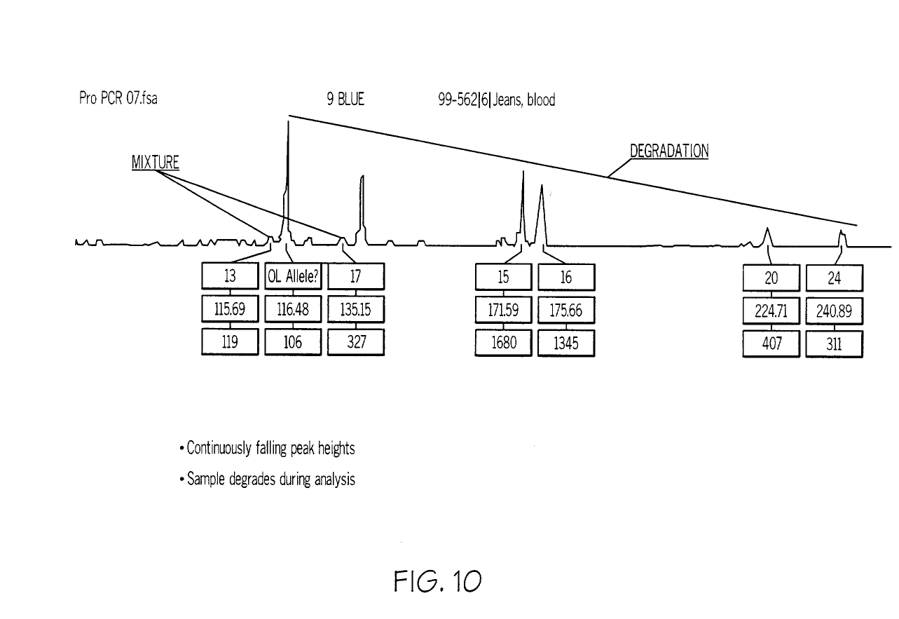

As illustrated by FIG. 10, degradation is indicated by diminishing peak heights from left to right. Degradation of the sample makes it very hard to determine the relevance of the peaks on the right because of the large change in peak height. The expert system is programmed to determine degradation when the peak heights from left to right are continuously diminishing, and if so, flags the peaks for later analysis by the user. In particular, degradation is determined in this embodiment by performing a linear regression on the peaks to find a fitted line. The slope of the fitted line is then determined and if negative, then peak height degradation for the sample is flagged by the expert system.

F. Stutter Peaks

Stutter peaks and noise are found in almost every electropherogram. Stutter peaks are small peaks that occur immediately before or after a true allele peak. They occur when the laser arise due to replication errors during the PCR amplification and creates a portion of a copy of the SIR allele. The expert system is programmed to determine stutter by applying stutter filters that discount peaks if they are less than 15% of the height of a peak that immediately follows them.

G. Noise

Noise is also present in all samples and constitutes the small peaks along the baseline. Sometimes noise can be a problem if its amplitude is similar to that of the signal from true alleles in a sample. Air bubbles, urea crystals, and sample contamination are among the possible causes of large peaks that are true technical artifacts that may mask contributions made by other contributors or may be mistaken as contributions made by a victim or suspect. The expert system is programmed to determine noise by looking for perturbations of the baseline that are not clearly associated with peaks from true alleles.

G. Raw Data Problems

A good indication of general analysis failure is to look at the raw data from the genetic analyzer. It should contain a large band followed by peaks that look similar to an output electropherogram graph of a convention genetic analysis program, such as ABI's GenoTyper program. The baseline should be constant. The expert system is programmed to determine raw data problems by performing the equivalent of a visual inspection for these attributes.

Additional rules and expert routines for making classifications of the received data may be determined by manual inspection of data, by interviewing experts in the problem domain, or by other knowledge acquisition techniques.

Alternatively, various machine learning methods may be applied to determine the rules, and optionally to determine rule confidence or accuracy. Rules may specify measures of uncertainty, as described above, which may be propagated to provide an overall confidence measure for each conclusion. Furthermore, the rules applied to reach a particular conclusion may be recorded so that the reasoning used to reach it may be explained to the user in text form. This is a facility usually provided by expert systems.

Benefits

The principal benefits of using the expert system 100 to perform analysis on forensic DNA evidence are in the ability to produce very high integration and complex, sophisticated program logic in a form that is easy for people to understand and extend. This is because the rules in the expert system can be stated in natural language (e.g., English), and because greater generality, flexibility, and accuracy can be obtained simply by adding new rules or modifying existing ones. The inference engine can then combine the rules to produce a degree of integration, sophistication, and thoroughness that is hard to reproduce by an orthodox procedural software approach.

The resulting output of the expert system 100 is the forensic DNA data and analysis summaries which are presented in a format useable to aid interpretation by both expert and non-expert humans, such as, for example, police officers, attorneys, judges, and juries. Example of such summaries, include an allelic summary table, which has a potential stutter in italics, victim in red, defendant in blue, and others in black, frequency calculations, and procedural interpretations. The procedural interpretations may include mixture indications, degradation indications, injection failure indications, peak height determination, marginal stutter indications, saturation determination, control check results, and the like.

The formatting of DNA data, frequency calculations, and procedural interpretations result from rules for determining which hypothesis about the DNA data is supported by the most evidence. Accordingly, the present invention may be used to generate easily interpreted files, objectively applies analysis parameters to all samples, provides a fast turn around of analysis, and efficiently draws attention to problems requiring further review.

### SUMMARY

The above described analysis routines of the present invention provide a method of screening the DNA output data of conventional genetic analyzer and analysis programs, flagging possible and probable problems associated with particular alleles and setup/run parameters in the received DNA data, which then can be evaluated later by an expert. It is to be appreciated that the output results of the present invention indicating these flagged problems are not meant as a definitive judgment, but only as a guideline. Accordingly, experts and non-experts will be able to look at only the areas of the analysis that are questionable. Less time will be spent reviewing the reliable aspects of an analysis, allowing for a more efficient and thorough review of possible problems.

Having described preferred embodiments of the present invention it will now become apparent to those of ordinary skill in the art that other embodiment and variations of the presently disclosed embodiment incorporating these concepts may be implemented without departing from the inventive concepts herein disclosed. Accordingly, the invention should not be viewed as limited to the described embodiments but rather should be limited solely by the spirit and scope of the appended claims.

What is claimed is:

1. A method for performing triage analysis of forensic DNA evidence on a computer, the method comprising:
obtaining a signal produced by a sequence of electrophoretically separated DNA fragments from the forensic DNA evidence, said signal providing allele information;
utilizing an expert system including a knowledge base and an inference engine to perform triage analysis on the DNA fragments using said signal, said inference engine executing at least one rule from said knowledge base for classifying said allele information; and
outputting summaries in a format useable to aid interpretation of the triage analysis.

2. The method according to claim 1 wherein said signal is obtained from the output of a genetic analysis program.

3. The method according to claim 1 wherein said allele information includes locus, allele labels, allele peak heights, allele peak areas, run type, and dye name.

4. The method according to claim 1 wherein said knowledge base comprises rule sets for characterizing low peak height, peak height degradation, peak height imbalance, spikes, mixtures, mixtures in positive controls, peaks in negative controls, gender error, injection failure, locus naming, population analysis, and combinations thereof.

5. The method according to claim 1 wherein said summaries include at least one of an allelic summary table, which has a potential stutter in italics, victim in red, defendant in blue, and others in black, frequency calculations, and a plurality of procedural interpretations.

6. The method according to claim 5 wherein said plurality of procedural interpretations includes providing population statistics, peak height determinations, mixture imbalance indications, degradation indications, injection failure indications, marginal stutter indications, saturation determination, control checks results, and combinations thereof.

7. The method according to claim 2 further comprises providing a communications link between said output of said genetic analysis program and said expert system in order to receive said signal.

8. The method according to claim 7 wherein said communications link is selected from at least one of a bus, peripheral connection, a network (private and/or public), landlines, wired, and/or wireless connections.

9. The method according to claim 2 wherein said output of the genetic analysis program is raw DNA detection data or electropherogram data.

10. The method according to claim 1 wherein said signal is provided on a computer-readable medium.

11. The method according to claim 10, wherein said computer readable medium includes at least one of a flash memory, CD, DVD, floppy, removable hard drive.

12. The method according to claim 1, wherein said summaries are outputted in either hardcopy or electronic form.

13. A software system for configuring a computer system comprising a processor, and a memory device, for performing triage analysis of forensic DNA evidence, the software system comprising program instructions for:
execution of a pre-processing routine which obtains as input a signal produced by a sequence of electrophoretically separated DNA fragments from the forensic DNA evidence, and prepares said signal for analysis;
execution of an automated analysis routine comprising a knowledge base and an inference engine to perform triage analysis on the DNA fragments using said signal, said inference engine executing at least one rule from said knowledge base for classifying allele information provided in said signal; and
execution of a post-processing routine which creates summaries in a format useable to aid interpretation of the triage analysis.

14. The software system according to claim 13 wherein said pre-processing routine checks the format of signal and converts said allele information contained in said signal to a useable format to analyze such data with said automated analysis routine.

15. The software system according to claim 14 wherein said converting said allele information renames files contained in said signal such that it has proper extension and read/write attributes for said automated analysis routine.

16. The software system according to claim 13 wherein said pre-processing routine saves files contained in the signal each to a portable document file in a directory, the directory containing modified files and a text file containing timestamp information for each file received in the signal, a size standard and analysis parameters used for each analysis run performed on the electrophoretically separated DNA fragments to create the signal.

17. The software system according to claim 16 wherein the timestamp information includes a directory creation date, a directory modification date, and a directory last accessed date.

18. The software system according to claim 13 wherein the automated analysis routine further comprises programming mimicking actions of an expert performing computerized analysis tasks on the forensic DNA evidence, wherein said action is performed using implemented timers.

19. The software system according to claim 13 wherein the post-processing routine creates summaries in hypertext-markup language allowing access to all files used and generated by the forensic analysis.

20. The software system according to claim 13 wherein at least one of said summaries is organized by run name, analysis type, and cutoff threshold.

21. The software system according to claim 13 further including program instructions for a front-end graphical user interface (GUI) providing options initializing the automated analysis routine to run on the allele information provided in the signal.

22. The software system according to claim 21 wherein said options include selecting a directory, creating a new directory, selecting files from analysis, selecting parameters, selecting analysis type, and selecting the initiation of the automated analysis routine.

23. The software system according to claim 13 wherein said summaries provide analysis parameters for all samples, analysis results for all samples, and analysis results for individual samples, each summary having a “Screen Shot”, “Size Standard”, and “Parameter” hypertext markup language buttons for retrieving and viewing the analysis parameters used for the samples.

24. The software system according to claim 23 wherein said “Screen Shot” hypertext markup language button if selected, opens an image of a machine default y-axis graph of corresponding dye colors (blue, green, yellow, red) for the DNA fragments, which allows a user to quickly view different locations of the DNA fragments.

25. The software system according to claim 24 further including a zoomed electropherogram which enables the user to analyze the machine default y-axis graph for the corresponding dye colors more closely.

26. The software system according to claim 23 wherein selection of said “Size Standard” hypertext markup language button opens a window which displays a size standard file, and selection of said “Parameters” hypertext markup language button opens a window which displays all of size information, RFU cutoff, and smoothing options selected for each analysis run contained in the signal.

27. The software system according to claim 13 further comprising program instruction permitting reanalysis of raw data provided in said signal using a different cutoff threshold to produce a second electropherogram having additional allele labels than a first electropherogram provided in the raw data.

28. The software system according to claims 13 wherein said allele information is grouped by samples and said summaries include hypertext markup language buttons used in viewing the analysis results for each one of the samples.

29. The software system according to claim 28 where said hypertext markup language buttons includes a “Result” button, which when selected for a particular sample opens a window which provides procedural interpretations regarding the particular sample, an “Info” button, which when selected for a particular sample opens a window which shows settings of a genetic analyzer used to provide the signal, a “Curve” button which when selected for a particular sample opens a window which shows how well the particular sample matches a size standard, a “Raw” button which when selected for a particular sample opens a window which shows unfiltered data contained in the signal from the genetic analyzer, and an “EPT” button which when selected for a particular sample opens a window showing voltage current, laser power, and temperature readings of the genetic analyzer during the run on the particular sample.

30. The software system according to claim 13 wherein the signal is output data from a conventional genetic analyzer, and said allele information includes peak height, peak area, sample type, size standard, dye name, and run type.

31. The software system according to claim 13 wherein said classifying the allele information includes detection of possible low peak height, detection of possible peak height degradation, detection of possible peak height imbalance, detection of a possible spike, detection of a possible mixture, detection of a possible mixture in a positive control, detection of possible peaks in a negative control, detection of a possible gender error, detection of a possible injection failure, locus naming, and population analysis.

32. An expert computer system for performing triage analysis of forensic DNA evidence, the computer system comprising:
means to load electrophoretic data containing a plurality of alleles,
means to format the data if necessary,
means to analyze said data, and
means to output results of analyzed data, said results providing indications of possible and/or probable problems corresponding to particle alleles in said data.

33. The expert computer system according to claim 32 wherein said means to load includes means to read said electrophoretic data from a file, or obtaining said data in real-time.

34. The expert computer system according to claim 32 wherein means to analyze said data includes means to scan with an inference engine program a list of rules, said program providing said results.

35. The expert computer system according to claim 34 wherein the list of rules are either forward chaining or backward chaining.

36. The expert computer system according to claim 32 wherein the means to analyze the data includes programming to instruct the expert system name locus and to count each allele peak provided in the data.

37. The expert computer system according to claim 32 wherein the means to analyze the data includes programming to indicate a possible mixture by instructing the expert system to check the number of peaks present in each locus, and to flag each locus if more than two alleles are present in one locus.

38. The expert computer system according to claim 32 wherein the means to analyze the data includes programming to indicate which alleles have a possible peak height imbalance by instructing the expert system to find a first allele within each locus, store a peak height of the first allele, finding a second allele in the same locus, store a peak height of the second allele, and if the peak heights of the first and second alleles differ by more than 30%, indicating the locus of the first and second alleles as having a possible peak height imbalance.

39. The expert system according to claim 32 wherein the means to analyze the data includes programming to indicate which alleles are noise by instructing the expert system to search the alleles in the data and tog each alleles probably noise if having a peak height of less than 50 RFUs, and to flag each allele is possible noise if having a peak height of less than 150 RFUs but greater than 50 RFUs.

40. The expert system according to claim 32 wherein the means to analyze the data includes programming to indicate a possible negative control problem if the expert system detects a peak height in the data labeled as a negative control.

41. The expert system according to claim 32 wherein the means to analyze the data includes programming to indicate mixtures by instructing the expert system to detect right and left alleles existing at the same locus and to flag the locus as a probable mixture if a peak of the left allele is less than or equal to 70 percent of a peak of the right allele, and to flag the locus as a possible mixture if the peak of the left allele is greater than 70 percent of the peak of the right allele and less than or equal to 90 percent of the peak of the right allele.

42. The expert system according to claim 32 wherein the means to analyze the data includes programming to indicate a positive control problem by instructing the expert system to check that only the expect alleles are present for a positive control, and to flag the positive control as having a possible positive control problem if a mixture or inappropriate alleles detected.

43. The expert system according to claim 32 wherein the means to analyze the data includes programming to indicate a spike by instructing the expert system to compare two alleles in a locus, and to flag each allele as a possible spike if the two alleles have the same peak height but differ in peak area.

44. The expert system according to claim 32 wherein the means to analyze the data includes programming to indicate a gender problem by instructing the expert system to check an amelogenin locus, and to flag the amelogenin locus as having a possible gender problem if a Y but no X alleles or if three alleles are found at the amelogenin locus.

45. The expert system according to claim 32 wherein the means to analyze the data includes programming to cluster alleles flagged as being part of a possible mixture by instructing the expert system to group alleles into two pairs based on similar peak height if four peaks are detected, and to cluster together the two alleles that are most closely correlated if three peaks are detected.

46. The expert system according to claim 32 wherein the means to analyze the data includes programming to indicate degradation by instructing the expert system to detect if two alleles exist in the same locus, and to provide the indication of a possible degradation at the DNA within a sample if the peak height of the left allele is greater than the peak height of the right allele.

47. The expert system according to claim 32 wherein the means to analyze the data includes programming to indicate injection failure by instructing the expert system to compare peak heights in a ROX sample from the data, and to flag a possible injection failure if peak heights are not very similar.

48. The expert system according to claim 32 wherein the means to analyze the data include programming assigning probabilities of racial derivation by instructing the expert system to use empirical data on a probability of a given locus existing in a racial group to determine a probability that a given sample came from the racial group, based on all of the alleles in the data.

49. The expert system according to claim 32 wherein the means to analyze the data include programming classifying single peaks as good by instructing the expert system to flag each peak in the data as a true allele peak if: Peak Area>(−2.1*peak height)+3600.

50. The expert system according to claim 32 wherein the means to analyze the data include programming identifying spikes in the data by instructing the expert system to flag each peak in the data as a spike if: Peak Area/Peak Height>10.

51. The expert system according to claim 32 wherein the means to analyze the data include programming to indicate degradation by instructing the expert system to perform a linear regression on peak heights within a locus, and to flag peak height degradation for the locus if a slope of a fitted line is less than negative 8.
